(19) United States
(12) Patent Application Publication
(10) Pub. No.: US 2004/0236258 A1
(43) Pub. Date: Nov. 25, 2004

(54) Inflatable support, kit and method

(76) Inventors: Michael Burns, Laconia, NH (US); Paul Burns, Cranston, RI (US); Gray Burns, Cranston, RI (US)

Correspondence Address: Lawson & Persson, P.C., Suite 103, 67 Water Street, Laconia, NH, 03246, US

(21) Appl. No.: 10/441,542

(22) Filed: May 20, 2003

(51) Int. Cl. A61F 5/00

(52) U.S. Cl. 602/13

(57) ABSTRACT

An inflatable support, kit of parts for forming a system for immobilizing a human leg, and a method of preparing an inflatable bag for supporting a human leg. The inflatable support includes a bag that has a single body having a first surface and a second surface. The body of the inflatable bag is made up of multiple air chambers running generally throughout. Portions of the bag between adjacent air chambers of the body form joined regions, such as notches, that allow the inflatable bag to bend to conform to the contours of a human leg. The inflatable bag body has a valve attached thereto so as to allow an inflation medium to pass into one or more of the air chambers.

### FIELD OF THE INVENTION

The present invention relates to a device and brace system for supporting a human limb, and in particular to an inflatable bag and a brace system accommodating such an inflatable bag for cushioning a limb.

### BACKGROUND

Inflatable ankle braces have been used for many years in the management of certain injuries to the lower extremities. These braces, commonly referred to as air casts, are typically utilized after immobilization of the extremity by a molded plastic or resin cast has stabilized the injury to a point where some weight may be placed upon the extremity.

Air casts typically include substantially rigid right and left outer shell members that are joined at the bottom by a flexible stirrup and shaped to fit a patient's ankle. The inside surfaces of these shell members are typically padded in selected areas and include some means for attaching an inner air cell or bladder. These air cells or bladders may include one or more compartments that may be inflated by the patient, or may be factory inflated. In addition, some such air casts include one or more compartments filled with a cushioning gel and/or multiple layers of open cell foam to provide additional padding.

Inflatable air casts have traditionally been inflated via a manual inflation tube that extends into the air bladder and is pressurized by the patient in a manner similar to the inflation of a balloon. Once pressurized, the tube is removed and the end sealed to prevent the escape of air. This inflation system is relatively inexpensive and provides for adequate inflation in many instances. However, it is not without its drawbacks. For example, patients having reduced lung capacity, due to age, respiratory disease or the like, may not be able to provide sufficient pressure into the tube to adequately inflate the bladder. In addition, air may escape during withdrawal of the tube, further reducing the pressure within the bladder. Also, the use of a tube does not allow for selective addition/removal of air to reduce pressure in the event that the bladder has been under-pressurized or over-pressurized. Finally, the tubes used to inflate the bladders in these systems are not readily adapted to being transported in a patient's pocket and, therefore, are not likely to be taken with the patient outside of the patient's home.

Bracing systems that facilitate inflatable bags with more convenient inflation means are beginning to emerge. For example, the inventors' issued U.S. Pat. No. 6,511,449, describes an inflatable brace system for immobilizing a lower extremity of a human. Aspects of this system involve multiple inflatable air bags configured within a brace to cushion and support a lower extremity of a human and a unidirectional air pump. The inflatable air bags are designed to be pressure controlled by simple unidirectional pumps. While such a multiple bag system may account for pressurization simplicity and brace comfort, other practical aspects are disregarded. A bracing system that contains multiple bags is more likely to exhibit a non-uniform pressure against an injured limb. Furthermore, manufacturers typically seek to minimize functional parts of an apparatus for increased construction efficiency, decreased material expenditures and attachment simplicity.

Therefore, there is a need for an inflatable bag for cushioning a human limb capable of simple inflation and deflation, convenient patient transport, providing substantially uniform pressure about a limb, simplified attachment to a bracing system, a more efficient construction that minimizes material costs, and a brace system capable of utilizing an inflatable bag with such attributes.

### SUMMARY

The present invention is an inflatable support, a kit of parts for a system for immobilizing a human leg, and a method of preparing an inflatable bag for supporting a human leg. In basic form, the inflatable support includes a bag that has a single body having a first surface and a second surface. The body of the inflatable bag is made up of multiple air chambers running generally throughout. Portions of the bag between adjacent air chambers of the body include joined regions, such as notches, that allow the inflatable bag to bend to conform to the contours of a human leg. The inflatable bag body further has a valve positioned so as to allow an inflation medium to pass into one or more of the air chambers. A preferred embodiment of the inflatable support includes a check valve located on the periphery of the body and allows air to pass to interconnected air chambers. A preferred body shape has two branches extending therefrom to form a groove sized to receive a human heel.

The basic kit of parts for a system for immobilizing a human leg includes the inflatable support and a stabilizing brace. The stabilizing brace envelops a substantial portion of a human leg and acts to prevent undesirable lateral motion of the internal components of the leg. The preferred stability brace for use with a leg is a walking boot, sometimes referred to as a stability boot. Such devices currently exist, and the preferred device is essentially similar to those already commercially available to support an injured leg. However, the stability brace of the inflatable brace system is further adapted to removably hold the unitary inflatable bag against a substantial portion of a human leg. Such removable holding is preferably achieved through the use of a hook and loop fastener system arranged on the second surface of the body of the inflatable bag and the inner surface of the stabilizing brace. However, other art-recognized means, such as buttons, snaps, or the like, may be substituted to achieve similar results. Further, it is recognized that the bag may be permanently attached to the boot via adhesives, stitching, mechanical fasteners, or the like.

A preferred version of the kit of parts further includes a unidirectional air pump dimensioned for attachment to the valve and selectively inflating and/or deflating the air chambers of the inflatable bag body. The preferred air pump has an inflation orifice and a deflation orifice and a bulb portion that causes air to be drawn in through the deflation orifice and out though the deflation orifice when squeezed by a user.

The basic method of preparing an inflatable bag for cushioning a human limb includes the steps of obtaining at least one sheet of heat fusible material, cutting the sheet to a size sufficient to wrap about a human leg when fused together to form a bag, fusing the sheet or sheets together to form an enclosed cavity having multiple interconnected air chambers, positioning a valve in contact with the cavity, and securing the valve in airtight relation with the cavity. The preferred method uses PVC sheets that are welded together using radio frequency energy. In this method, uncut sheets are placed between a platen having the desired shape and joining details, and a horn, which vibrates at high frequency and causes the material to be pressed therebetween to melt and fuse together. In this preferred method, the cutting step is performed by the die concurrent with the fusing step.

In some embodiments, the method further involves the partial filling of one or more of the air chambers of the cavity with either atmospheric air or a substantially viscous material, such as gel.

Therefore, there it is an aspect of the invention to provide an inflatable support capable of comfortably bracing and cushioning a human leg.

It is a further aspect of the invention to provide an inflatable support capable of simple inflation and deflation.

It is still a further aspect of the invention to provide an inflatable support capable of convenient patient transport.

It is still a further aspect of the invention to provide an inflatable support capable of providing substantially uniform pressure about a limb.

It is still a further aspect of the invention to provide an inflatable support capable of simplified attachment to a bracing system.

It is still a further aspect of the invention to provide an inflatable support capable of more efficient construction that minimizes material costs.

It is still a further aspect of the invention to provide an inflatable support with minimal leakage of an internal inflation medium.

It is still a further aspect of the invention to provide an inflatable brace system that includes a means of inflation and deflation that allows people with reduced respiratory capacity to adequately inflate the air bladder.

It is still a further aspect of the invention to provide an inflatable brace system that includes a means of inflation and deflation that allows the bag to be selectively inflated and deflated.

It is a still further aspect of the invention to provide an inflatable brace system that includes a means of inflation and deflation that is relatively inexpensive.

It is a still further aspect of the invention to provide an inflatable brace system that includes a means of inflation and deflation that is easily transported by a patient.

These aspects of the invention are not meant to be exclusive and other features, aspects, and advantages of the present invention will be readily apparent to those of ordinary skill in the art when read in conjunction with the following description, and accompanying drawings.

### BRIEF DESCRIPTION OF THE DRAWINGS

FIG. 1 is a front isometric view of one embodiment of the inflatable bag of the present invention.

FIG. 2 is a rear isometric view of the preferred inflatable bag of the present invention.

FIG. 3 is a cut away side view of the valve of the preferred embodiment of the inflatable bag of the present invention.

FIG. 4 is an exploded schematic perspective view of one embodiment of the system of the present invention.

FIG. 5 is an exploded schematic perspective view of another embodiment of the system of the present invention used as a walking-boot type brace.

FIG. 6 is a front view of one embodiment of the inflatable bag of the present invention in which the body includes a gel pack.

FIG. 7 is a front view of the valve and unidirectional pump of the preferred kit of the present invention.

FIG. 8 is a front view of an alternative embodiment of the brace in which the inflatable bag has a substantially firm backing.

### DESCRIPTION OF THE DRAWINGS

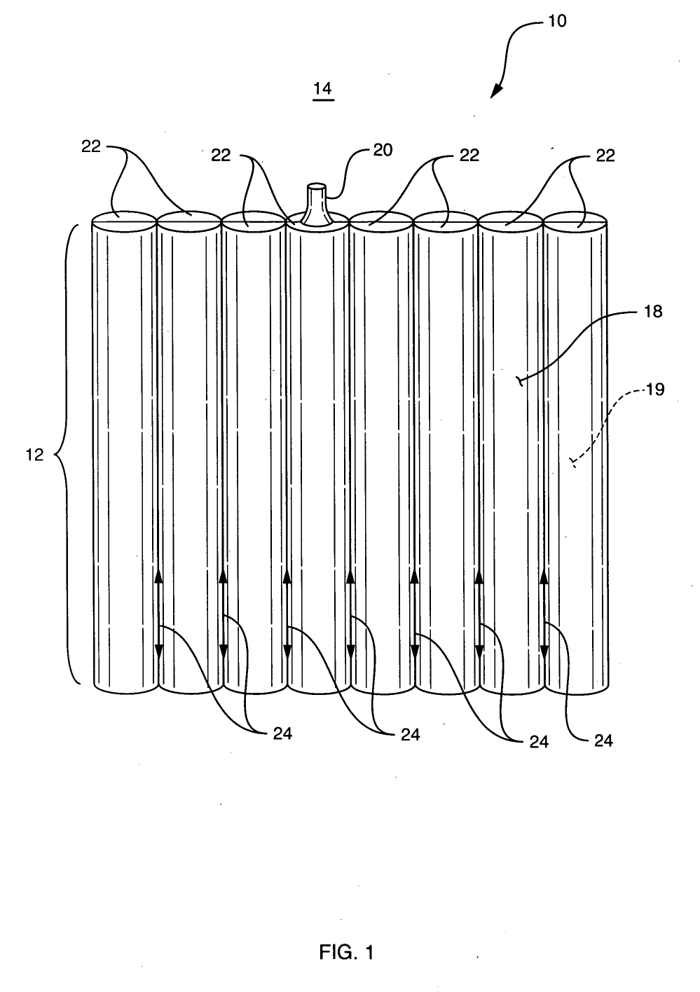

Referring first to FIG. 1, a basic embodiment of the inflatable support 14 is shown. The inflatable support has an inflatable bag 10 with a unitary body 12. The unitary body 12 has a first sheet 15 having a first surface 18 and a second sheet 17 having a second surface 19. First sheet 15 and second sheet 17 may be separately cut sheets of a suitable material, or may be a single sheet folded upon itself such that the fold defines the first sheet 15 and second sheet 17. The body 12 of the inflatable bag 10 of FIG. 1 is made up of multiple air chambers 22 running generally throughout the body 12. The first sheet 15 and second sheet 17 are joined together at locations along the body to form joined regions, here in the form of notches 24 that define the air chambers 22 and allow the bag 10 to conform to a human leg. Additionally a valve 20 connects to the air chambers 22 to allow air to be inserted into, and withdrawn from, the chambers 22.

The body 12 of the inflatable bag 10 is a single unit having more than one air chamber 22. For descriptive purposes, the body 12 can be viewed in various sections: a first sheet 15 having a first surface 18 and a second sheet 17 having a second surface 19. The first surface 18 is the surface that would contact a human limb in preferred embodiments. The second surface 19, opposite from the first surface 18, preferably serves as a point from which to place attachment means, if such means are desirable. This version of the invention exhibits the preferred location of a valve 20, on the periphery of the body 12. However, it is recognized that other locations could be used to achieve similar results.

The air chambers 22 of the body 12 serve as repositories for inflation media. The air chambers inflate or deflate in response to pressurization. As the name suggests, the air chambers 22 are preferably filled with air; presumably from the atmosphere, though any stable gas would expand an air chamber 22 and achieve the same result as atmospheric air. Other versions of the present invention might facilitate alternative inflation media such as gel, liquid or other substantially viscous materials. However, it is preferred that atmospheric air is used to inflate and deflate the body 12 so as to allow simple, widely-available pumps to be used in conjunction with the bag 10.

The body 12 is preferably constructed of durable heat-sealable plastic or similar material that can expand repeatedly without affecting the material's integrity. It is further preferred that each air chamber 22 has at least a small amount of interconnection to all other air chambers 22 so as to allow a single pressurization source to fill each air chamber 22 of the body 12 simultaneously. Interconnecting air chambers 22 prevent the need for multiple pressurization passages, i.e. valves 20, openings or the like, and provide a uniform pressure within all chambers 22 of the bag 10.

In the preferred embodiment, notches 24 form the sealed borders of the air chambers 22, which serve to make each air chamber 22 at least partially distinct from its adjacent air chamber 22. The notches 24 span much of the distance between bordering air chambers 22, but will likely have lessor lengths than the air chambers 22 due to the previously mentioned interconnection of the preferred air chamber 22. The advantage of the notches 24 is the resulting flexibility of the body 12. Because the notches 24 are of relatively negligible thickness, the body 12 may contort to fit about an extremity of a human, such as a leg, arm, or the like. Notches 24 running almost throughout the entire length of the body 12 are preferred as these provide for maximum flexibility of the body 12. However, it is recognized that other joined areas would provide similar results. For example, one or more circular or oval joined areas at desired locations along the sheets 15, 17 would likewise allow the bag to conform to a human limb. Accordingly, the inflatable bag 10 of present invention should not be seen as being limited to bags having the preferred notches 24.

The valve 20, shown as attached the periphery of the body 12, serves as a passage for gas pressurization of one or more air chambers 22. Air chamber 22 interconnection would obviate the need for more than one valve 20, and such interconnection in conjunction with a single valve 20 is preferred. It is recognized that alternate versions of the present invention having non-interconnected air chambers 22 might be desirable, but would require an extra valve 20 for each totally distinct airflow barrier. The valve 20 may be any opening that leads from the atmosphere, or other inflation source, to at least one air chamber 22 and is then capable of preventing inflation medium egress. It is preferred that that valve 20 be disposed upon the body 12 of the bag 10 such that it is accessible for inflation or deflation when worn by the user. However, in other embodiments, such as those for use with children, the valve 20 is located in a location that is not readily accessible when the accompanying bracing system is in use.

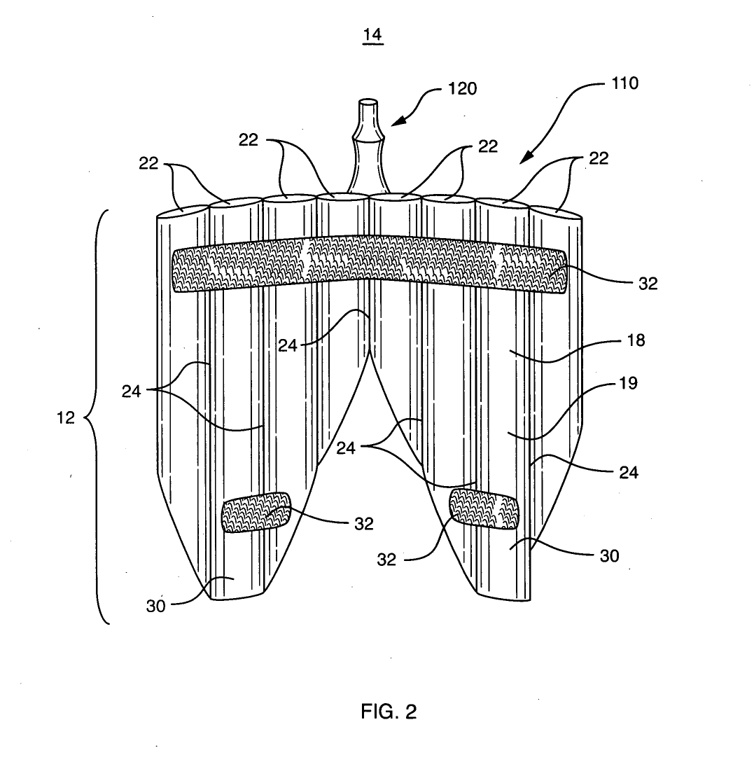

Now referring to FIG. 2, the ankle-compatible bag 110 is shown. This embodiment exhibits the single body 12 construction of the present invention, but features a shape more amenable to the natural contours of the lower extremity of a human leg. The body 12 includes two branches 30 that jut to form a void therebetween. When the ankle-compatible bag 110 wraps about the furthest extremity of a human leg, the branches 30 wrap about the ankle in order to account for the limited space of the common brace. The branches 30 do not necessarily have a preferred size due to the varied shapes and sizes of the human ankle. One sure solution to account for this size diversity is to simply manufacture ankle-compatible 110 bags in varied sizes, much like the footwear industry.

The air chambers 22 and notches 24 of the ankle-compatible bag 110 are sized to conform to the desired shape of the body 12. It is noted that neither the air chambers 22 nor the notches 24 of any embodiment necessarily have a preferred size or shape, and are adjustable to the particular area of the human extremity to which one desires to place the present invention.

FIG. 2 additionally exhibits the second surface 19 of the ankle-compatible bag 110, with the preferred attachment means bonded to it. The preferred attachment means is made up of layers of felt 32 designed to mate with layers of hook and loop fastener material, such as that commonly sold under the VELCRO® trademark. Alternatively, the hook and loop fastener could be placed directly upon the second surface 19. For added stability and attachment ease, the entire second surface 19 might be covered with either hook and loop fastener or a material sufficient to mate with it such as some version of a felt material or the like. A hook and loop fastener bound to the body 12 would be advantageous as it would allow the bag 110 to be removed from a brace quickly and nearly effortlessly while still allowing for solid attachment to a hook and loop fastener compatible brace. The hook and loop fastener or its compatible mating material need not be bound to the second surface 19 of the body; it could be attached by side straps or the like. Other art recognized means, such as buttons, snaps, mechanical fastners, or the like may be employed to removably attach the ankle-compatible bag 110 to a brace. Further, the bag 110 may be permanently attached to the brace via an adhesive, stitching, or permanent mechanical fasteners, such as rivets, to produce similar results.

The first surface 18 of the body 12 serves to contact human skin. Generally, the first surface 18 would simply be the bare material used in fabricating the air chambers 22. However, other materials could be placed about the first surface 18 so as to act as a buffer between the body 12 and human skin. For example, the preferred embodiment uses brushed tricot, which is flame laminated to a PVC inner core. Alternatively, cotton or other materials compatible with the hook portions of hook and loop fasteners could be substituted to achieve similar results. It is recognized, however, that a vast multitude of padding materials exist and that any of these art recognized padding material might be used on the first surface 18.

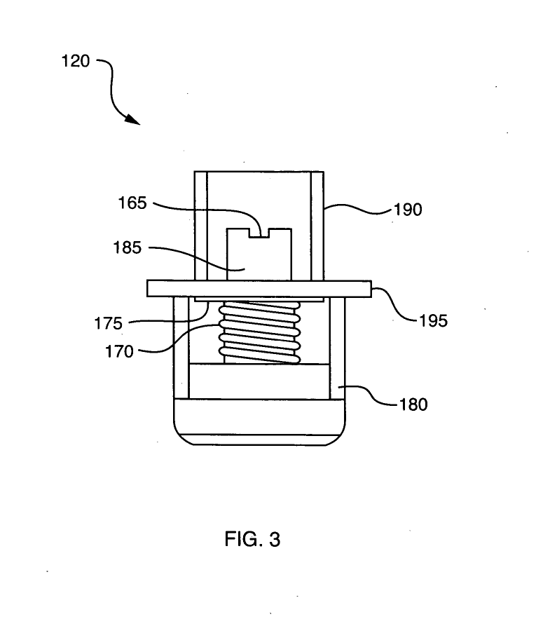

This ankle-compatible bag 110 embodiment further features the preferred valve: a check valve 120. The preferred check valve is examined in more detail in FIG. 3 and includes a spring-loaded valve 120 that is sealed in such a manner as to prevent air leakage when the inflatable bag is inflated. Valve 120 includes a top portion 190 that extends above the plies of the inflatable bag 10, a shoulder portion 195 that is dimensioned to sit atop the plies and prevent the valve from being pushed into the inflatable bag, and a lower portion 180 that is dimensioned to be heat sealed between the plies of the inflatable bag. However, it is recognized that the valve 120 may be attached at different locations and using different art recognized means. A spring 170 is mounted within the lower portion 180 and acts to bias a valve pin 185 upward to maintain the valve 120 in a closed position. The valve pin 185 includes a sealing ring 175 along its bottom surface for sealing off the valve when in the closed position, and a groove 165 along its top extremity for allowing air to flow into the inflatable bag 110 when the valve pin 185 is depressed.

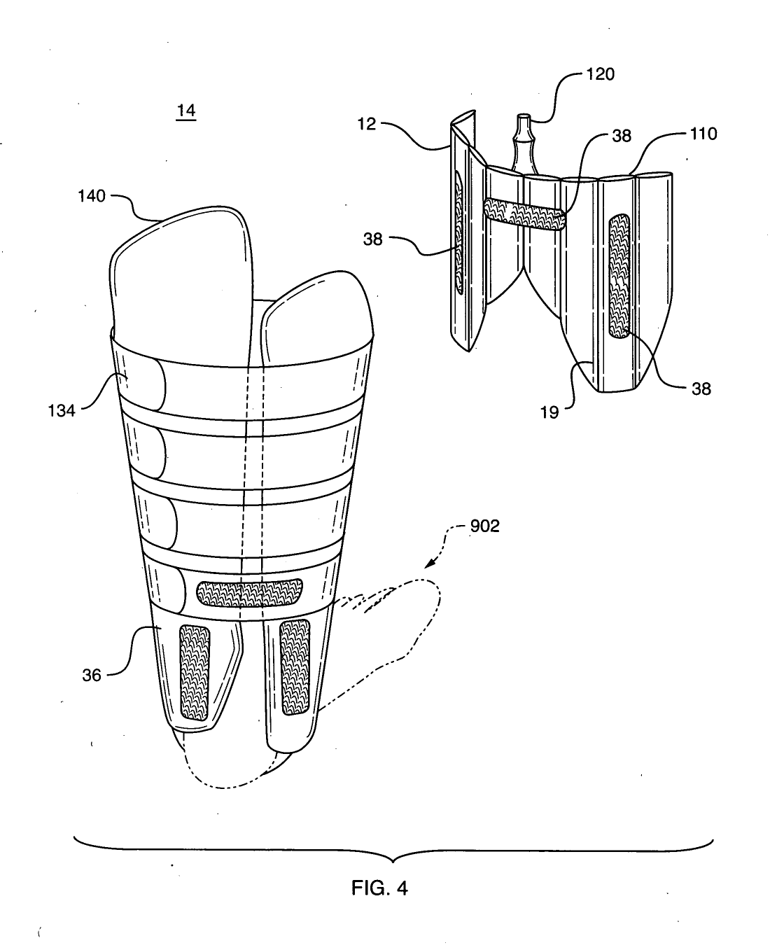

FIG. 4 shows the preferred inflatable bag 110 of FIG. 2 set to attach to a leg version of the inflatable brace system 140. The body 12 of the inflatable bag 110 wraps around a substantial portion of the leg. In operation, the inflatable bag 110 occupies a position between the leg and the stability brace 134. The stability brace 134 shown is a brace that fixes itself about a leg by use of straps and hoops sized to accommodate each other. This is merely illustrative of a common commercial version of a brace familiar to those in the art. Brands, sizes, weights and types of leg stability braces 134 are generally arbitrary to the present invention. The important attributes associated with a compatible leg version of the stability brace 134 are that it is capable of holding the leg, the inflatable bag 110 and itself 134 substantially stationary. The preferred means of holding the inflatable bag 110 stationary with respect to the leg version of the stability brace 134 is the use of mating hook and loop fastener components 38. Such components 38 are placed upon portions of the second surface 19 of the body 12 of the inflatable bag 110 and upon portions of the inside surface 36 of the leg version of the stability brace 134. However, as noted above, there are abundant ways of attaching the body 12 with respect to the leg version of the stability brace 134. As shown in FIG. 4, it is also advantageous, though not necessary, to position the valve 20 so as to provide a wearer access to it, even while the stability brace 34 is secured. Thus, positioning the valve 20 on an extremity of the bag 110 is preferred.

The inflatable bag 110 having branches 30 is preferably placed into the inflatable support 140 of FIG. 4. When placed into the leg version of the stability brace 134, the branches 30 would wrap about the human ankle and the resulting groove would adequately receive the human heel. However, the embodiments the inflatable bags are substantially interchangeable with any brace system embodiment.

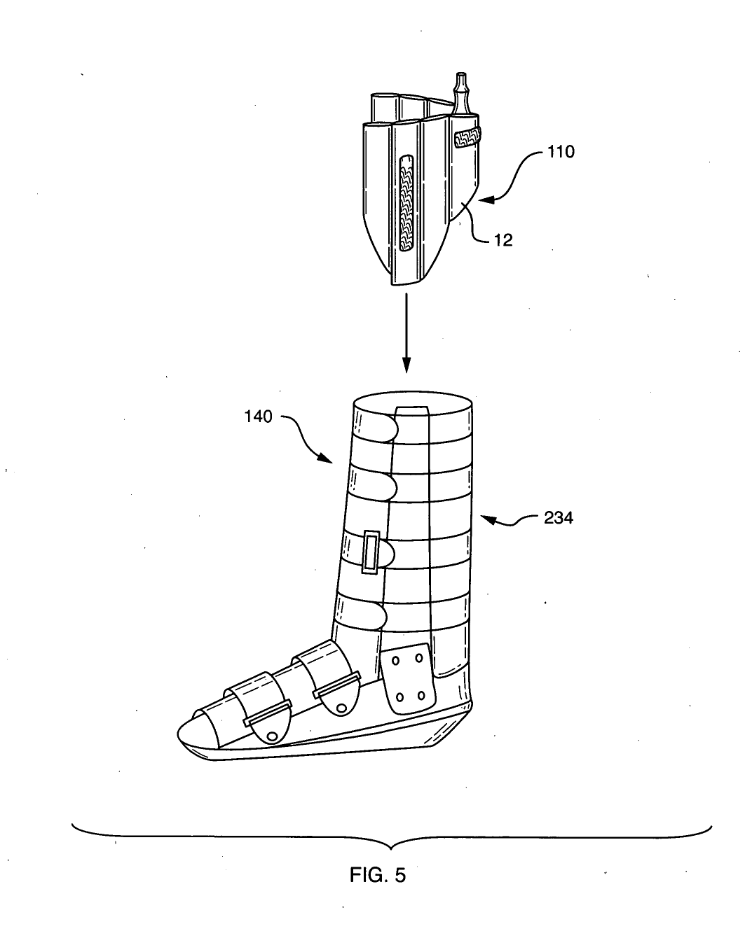

FIG. 5 shows the preferred inflatable bag 110 of FIG. 2 set to attach to the preferred leg version of the inflatable support 140. This alternative version uses a walking boot 234, sometimes called a stability boot, as the stability brace. Walking boots are widely available medical items distinguished from typical braces by having non-therapeutic aspects. While walking boots have the leg stabilizing features, they generally further have a foot cover and a sole often having a treaded surface. These additional elements allow a wearer the ability to use the walking boot as a substitute for a shoe.

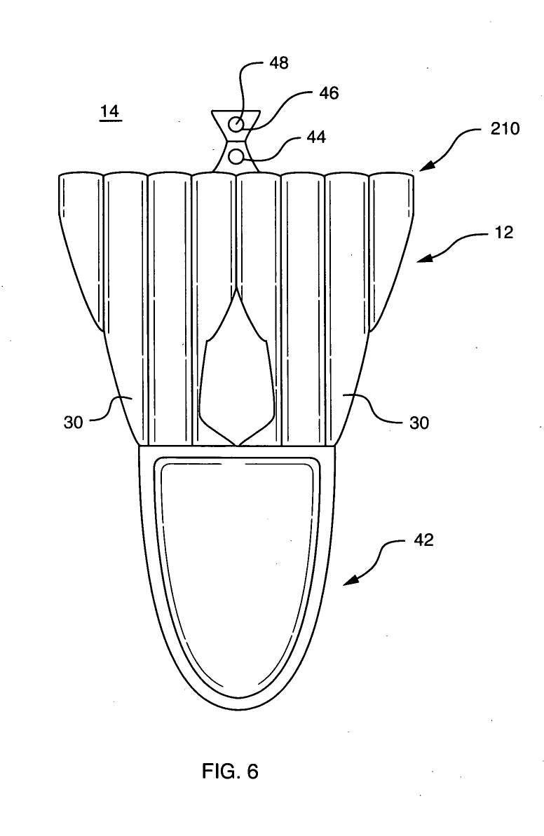

FIG. 6 shows an alternate version of the inflatable bag 210 having a body 12, branches 30, and an impact pad 42. The impact pad 42 is shaped so as to fit into a walking boot in a position beneath where a foot would rest. The preferred impact pad 42 is a gel pack, which is recognized in the art as being a particularly good absorber of shock. However, other art recognized means of comfortably padding a foot during use, such as open or closed cell foam, cotton batting, or the like, may be substituted.

The embodiment of the inflatable bag 210 of FIG. 6 also includes a foldable valve 46 for inflating at least one air chamber. Foldable valves are more common among pressurized cavities formed of multiple plies. Each ply includes an integral extension and end flap that forms a self-sealing foldable valve 46. This foldable valve 46 is adapted to seal any air in a first chamber formed interiorly of the joined plies, thus preventing loss of internal pressurization, and may be selectively opened as by inserting a suitably sized plastic tube between the confronting flaps to admit or exhaust air from the first chamber. A pair of mating hook and loop fasteners 44, 48 may be affixed to the valve exterior 46 so that the valve 46 may be bent upon itself and fastened in place.

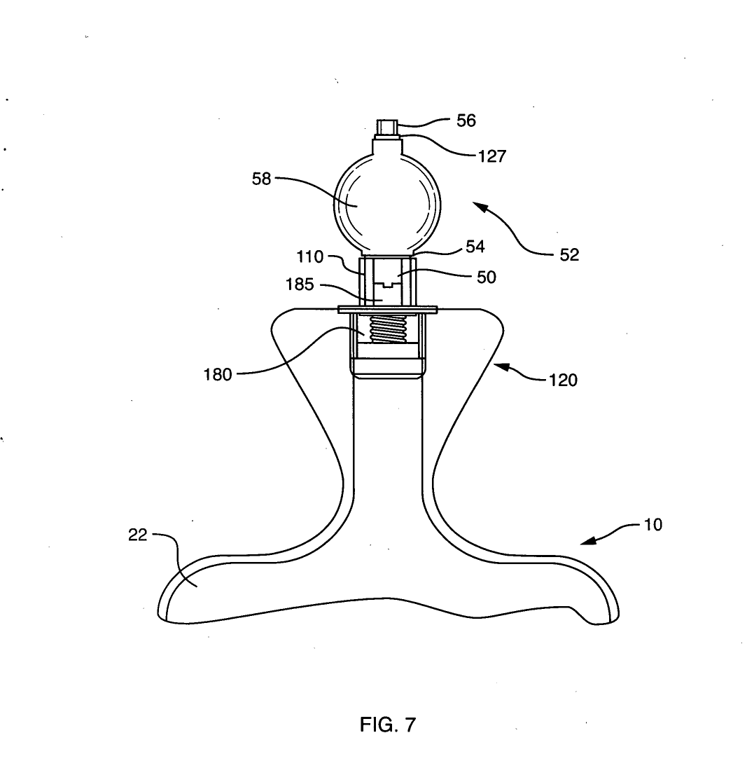

FIG. 7 displays the air pump of the kit of parts for a system for immobilizing a human limb. The inflatable bag 10 is inflated by inserting an inflation orifice 50 of a bulb type pump 52 into the top portion 190 of the valve 120. During inflation, the inflation orifice 50 depresses the valve pin 185 to allow air to create a fluid path between top portion 190 and the lower portion 180. The shoulder portion 54 of the pump 52 is then brought into contact with the rim of the top portion 190 such that air is prevented from escaping. Air is then pumped through the inflation orifice 50 into an air chamber 22 of the inflatable bag 10.

In these embodiments, the pump 52 is a dual bladder pump having an inflation orifice 50 and a deflation orifice 56. In such a pump 52, manually squeezing the bulb 58 of the pump causes air to be drawn into the pump 52 through the deflation orifice 56 and to be exhausted through the inflation orifice 50. Because of the unidirectional nature of the airflow through such a pump 52, this pump 52 is readily adapted to controllably deflate the inflatable bag 10 by simply inserting the deflation orifice 56 into the top portion 190 of the valve 120 and squeezing the bulb in a similar manner. In operation, the pump 52 is used to inflate the inflatable bag 10 before the patient attaches the brace about his lower extremity. The user will then adjust the pressure in the inflatable bag 10 by inserting the inflation orifice 50 or deflation orifice 56 of the pump into the top portion 190 of the valve 120 and squeezing the bulb 58 until the desired pressure is achieved.

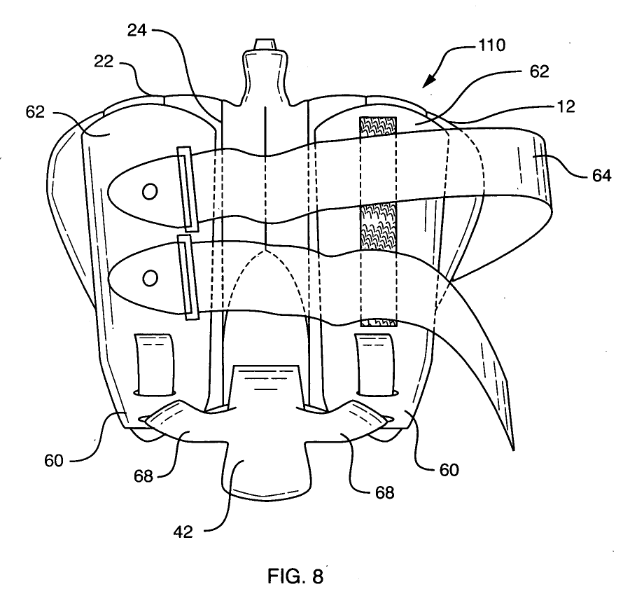

FIG. 8 shows a version of the inflatable bag 110 having a substantially firm backing 60. The substantially firm backing 60 serves as a protective hard outer covering for the inflatable bag 110. The substantial firmness of the backing 60 should be at least of magnitude to prevent deformation by prolonged use, to prevent inflatable bag 110 from being punctured by ordinary sharp objects (i.e. pencils, scissors and the like), and to allow the inflatable bag 110 to maintain a constant pressure against a leg. The preferred backing 60 is constructed of molded plastic and constitutes a pair of shell members 62 fastened to the body 12 of the bag 110. Though it need not be, such fastening is preferably permanent so as to increase durability, stability and longevity.

The preferred duo of shell members 62 is attached by at least one binding strap 64. Any binding straps 64 attach to at least one of the shell members 62. Means for grasping binding straps 64 are located on another shell member 62. The means for grasping may involve hook and loop fasteners, string, buttons, or some other art recognized fastening system. Preferably, the binding strap 64 is fastened to a VELCRO®) strip disposed upon a surface of the shell member 62. The pictured version of the inflatable bag 110 further includes an impact pad 42 positioned between two shell members 62. The impact pad 42 may be fastened to either the inflatable bag 110, the backing 60 or any other surface proximate to the present invention while in use. The location, though not arbitrary, is merely a matter of design choice, convenience and structural necessity. The impact pad 42 is shaped to support the heel of a human. Furthermore, impact pad straps 68 join the impact pad 42 to the shell members 62 in manner that maintains the impact pad 42 in a position appropriate to support the heel.

The present invention likewise encompasses the method of making the inflatable bags described above. The first step in the method is to obtain at least one sheet of a heat sealable or fusible material. As noted above, this material is preferably PVC with brushed tricot flame laminated thereto. The sheet is then cut to a size sufficient to wrap about a human leg when fused together to form a bag. This may mean that it is cut into two sheets, or may be a single sheet that is folded upon it. The sheet or sheets are fused together to form an enclosed cavity having multiple interconnected air chambers. This fusing step may be done separate from the cutting step, although it is preferred that both the cutting and fusing steps be performed concurrently. Once fused, a valve is positioned in contact with the cavity and is secured in airtight relation therewith.

The preferred method uses PVC sheets that are welded together using radio frequency energy. In this method, uncut sheets are placed between a platen having the desired shape and notches and a horn, which vibrates at high frequency and causes the material to pressed therebetween to melt and fuse together. As noted above, in this preferred method, the cutting step is performed by the die concurrent with the fusing step. In the art, using radio frequency energy to seal objects is typically referred to as Radio frequency welding, also known as RF welding, dielectric welding, or high frequency welding. Radio frequency welding is used to join or assemble various thermoplastic materials such as PVC (polyvinylchloride), polyurethanes, and others. The resulting seam is impervious to fluids as no needle holes are created. PVC, in particular is an excellent medium for RF welding.

In embodiments using RF welding, a number of joined areas are formed in the cavity to allow the bag to bend to conform to a human limb and to substantially segment the cavity using into multiple interconnected air chambers. In the preferred embodiment, this is achieved by welding straight substantially parallel lines across a portion of the inner body of the inflatable bag. The RF welding will fuse the two sheets together forming the notches of the inflatable bag. Those notches will serve to create generally distinct air chambers. The preferred air chambers will not be totally distinct as the preferred air chambers are welded so as maintain some semblance of cavity interconnection. In cases where air chambers are totally distinct, then valves will need to be placed in fluid communication with each totally distinct each air chamber.

The positioning of the valve in contact with the air chambers of the cavity may be performed in a variety of ways. In cases of totally distinct air chambers, this might involve placing a valve in contact with each totally distinct air chamber. However, as a preferred version of the process involves RF welding so as to allow fluid interconnection between each and every air chamber, the preferred valve positioning step places a single valve in a location accessible to a wearer even in use. The pictured embodiments of FIGS. 1-8 exhibit an abutting centrally located valve on the periphery of the inflatable bag. The location of the valve upon the inflatable bag is generally immaterial to the invention as a whole. Due to air chamber interconnection, the valve may be placed on any surface or part of the inflatable bag and still achieve the prominent purpose of the bag: to fill the air chambers with a supporting inflation medium such as air, gas, gel or the like.

The valve might be any opening on or about any surface of the inflatable bag. While the preferred valve is a check valve, the valve of the present invention is any orifice allowing interaction between an air chamber and an exteriorly located pump. For example, simpler embodiments might utilize only a hole that is immediately sealed after use.

The method optionally involves filling the air chambers of the cavity with atmospheric air. Though it is acknowledged that any stable gas could be used in filling the air chambers of the present invention, atmospheric air is the preferred due to cost concerns. Furthermore, atmospheric air use increases ease of inflatable bag adjustment because one performing the process may usually have immediate access to atmospheric air. In the alternative, a substantially viscous material may be used to occupy the volume of the air chambers. A substantially viscous material is a material having a density greater than that of a room temperature gas, but less than that of a solid. A material having a gel consistency is the preferred viscous material. Gels have certain distinct advantages over air. Filling the inflatable bag with gel increases the support that the inflatable bag would pose with respect to an injured extremity. Furthermore, because gels have a greater density than a gas, a gel would be less able to seep through any cracks in the surface of the inflatable bag.

Another optional step of the method of the present invention includes removing the valve after filling the bag with a substantially viscous material. As previously mentioned, having the inflatable bag of the present invention filled with gel prevents seepage. Decreased seepage allows the inflatable bag to deviate less from its original support pressure. This decreased deviation might obviate the need to replace or augment the gel within the air chambers. Because the valve is the means for medium augmentation and replacement, there are certain advantages its removal. One such advantage is to further decrease potential inflatable bag leakage. Regardless of a valve's ability to check leakage, a valve is still includes a hole (here, the largest hole in the present invention). Not only are valves not perfectly efficient, but they are also capable of breakage. Removing the valve after filling with a substantially viscous material negates the greatest potential leakage problem.

Although the present invention has been described in considerable detail with reference to certain preferred versions thereof, other versions would be readily apparent to those of ordinary skill in the art. Therefore, the spirit and scope of the appended claims should not be limited to the description of the preferred versions contained herein.

What is claimed is:

1. An inflatable support for a human leg, said support comprising
an inflatable bag comprising a unitary body having a first sheet having a first surface and a second sheet having a second surface
wherein said body comprises at least one joined region in which said first sheet is joined to said second sheet, said at least one joined region being located and dimensioned to allow the bag to bend to conform to a human limb and to define two inflatable air chambers;
wherein said body is dimensioned to wrap about a substantial portion of a human leg when each of said air chambers is inflated; and
wherein said body is manufactured of a material that is capable of pressurization and sufficiently flexible to bend along each joined region to conform to a human limb; and
at least one valve in fluid communication with at least one of said inflatable air chambers.

2. The inflatable bag of claim 1 wherein each of said at least one joined region comprises a notch and wherein each air chamber is in fluid communication with all other air chambers.

3. The inflatable bag of claim 2 wherein said inflatable bag further comprises a substantially firm backing attached thereto.

4. The inflatable support of claim 3 wherein said backing includes at least two shell members stabilized by at least one binding strap.

5. The inflatable support of claim 4 wherein two of said shell members further include an impact pad attached therebetween, said impact pad dimensioned to accommodate at least a portion of a human heel.

6. The inflatable support of claim 2 wherein said body includes two branches defining a slot dimensioned to accommodate at least a portion of a human heel.

7. The inflatable support of claim 6 further comprising an impact pad joined to at least one of said branches.

8. The inflatable support of claim 7 wherein said impact pad is at least one gel pack.

9. The inflatable support of claim 2 wherein said inflatable bag further includes a fabric material at least partially covering at least one surface of said body.

10. The inflatable support of claim 9 wherein said inflatable bag further includes a fabric material at least partially covering both surfaces of said body.

11. The inflatable support of claim 2 wherein said inflatable bag is at least partially constructed of heat-sealable material.

12. The inflatable support of claim 2 further comprising a portion of a hook and loop fastener attached to one of said first surface and said second surface.

13. A kit of parts for a system for supporting a human leg, said kit comprising:
a stability brace having an inside surface;
an inflatable bag comprising:
a unitary body having a first sheet having a first surface and a second sheet having a second surface
wherein said body comprises at least one joined region in which said first sheet is joined to said second sheet, said at least one joined region being located and dimensioned to allow the bag to bend to conform to a human limb and to define two inflatable air chambers;
wherein said body is dimensioned to wrap about a substantial portion of a human leg when each of said air chambers is inflated; and
wherein said body is manufactured of a material that is capable of pressurization and sufficiently flexible to bend along each joined region to conform to a human limb; and
at least one valve in fluid communication with at least one of said inflatable air chambers.

14. The kit of claim 13 further comprising a unidirectional air pump dimensioned to be attached to said valve and selectively inflate and deflate at least one of said air chambers of said body.

15. The kit of claim 14 wherein said air pump has an inflation orifice and a deflation orifice.

16. The kit of claim 15 wherein said air pump has a bulb portion dimensioned to draw air in through said deflation orifice and out through said inflation orifice when squeezed by a user.

17. The kit of claim 15 wherein both at least a portion of said inside surface of said stability brace, and at least a portion of said second surface of said inflatable body have hook and loop fastener components fitted to mate therebetween.

18. The kit of claim 13 wherein each air chamber is in fluid communication with all other air chambers.

19. The kit of claim 13 wherein said body includes two branches defining a slot dimensioned to accommodate at least a portion of a human heel.

20. The kit of claim 19 further comprising an impact pad joined to at least one of said branches.

21. A method of manufacturing an inflatable bag for supporting a human leg, said method comprising the steps of:
obtaining at least one sheet of a heat fusible material;
cutting said at least one sheet to a desired size;
fusing said at least one sheet to form an enclosed cavity having a periphery and a plurality of interconnected air chambers;
positioning a valve in contact with said periphery of said cavity; and
securing said valve in airtight relation with the cavity;
wherein said desired size of said at least one sheet is a size sufficient to wrap about a human leg once said bag is inflated.

22. The method as claimed in claim 21;
wherein said at least one sheet is two sheets of a polyvinyl chloride material; and
wherein said welding step comprises welding said two sheets together using radio frequency energy.

23. The method as claimed in claim 22 wherein said cutting step is performed concurrent with said fusing step.

24. The method as claimed in claim 23 further comprising the step of injecting a fluid within said cavity.
